(19) United States
(12) Patent Application Publication
(10) Pub. No.: US 2025/0022597 A1
(43) Pub. Date: Jan. 16, 2025

(54) GENERALIZABLE MACHINE LEARNING MEDICAL PROTOCOL RECOMMENDATION

(71) Applicants: GE Precision Healthcare LLC, Waukesha, WI (US); The Board of Trustees of the Leland Stanford Junior University, Stanford, CA (US)

(72) Inventors: Debashish Pal, Sunnyvale, CA (US); Yaxi Shen, Waukesha, WI (US); Steven Nichols, Okauchee, WI (US); Ravi Raj Singh, Bokaro Steel City (IN); Amanda Ciano, Waukesha, WI (US); Arindam Dutta Choudhury, Bangalore (IN); Akshay Chaudhari, Stanford, CA (US); Andreas Loening, Stanford, CA (US); Curt Langlotz, Stanford, CA (US); Naeim Bahrami, San Diego, CA (US)

(21) Appl. No.: 18/713,563

(22) PCT Filed: Nov. 23, 2022
(86) PCT No.: PCT/US2022/050912

§ 371 Date: May 24, 2024

(30) Foreign Application Priority Data
Nov. 25, 2021 (IN) 202141054621

(51) Int. Cl. G16H 50/20; G16H 10/60; G16H 40/20; G16H 70/20

(52) U.S. Cl. CPC G16H 50/20; G16H 10/60; G16H 40/20; G16H 70/20

(57) ABSTRACT

An architecture and techniques for providing a generalizable machine learning recommendation in connection with medical protocols such as radiology protocols. In response to receipt of a medical examination order request in a standardized input format, the system can, based on a machine learning technique, output a recommended protocol according to a standardized output format. The system can then perform a mapping procedure that maps site-specific data to the standardized input format and the standardized output format. The site-specific data can comprise information that is specific to an entity that provides the medical examination order request.

### CROSS-REFERENCE TO RELATED APPLICATION

This patent application claims priority to, India Provisional Patent Application No. 202141054621, filed Nov. 25, 2021, and entitled “GENERALIZABLE MACHINE LEARNING MEDICAL PROTOCOL RECOMMENDATION,” the entirety of which is hereby incorporated by reference herein.

### TECHNICAL FIELD

This disclosure relates generally to utilizing machine learning techniques to provide a medical protocol recommendation and more specifically to techniques that enable the recommendation to be generalizable to specific entities such as specific hospitals.

### BACKGROUND

When doctors consult with patients, imaging examination procedure (e.g., a computerized tomography (CT) scan) is frequently requested to learn more about the condition of the patient. These imaging examination procedures are initiated by a medical doctor (or another suitable healthcare professional) filling out an imaging examination order request required to schedule and perform imaging services for a patient. In that regard, the imaging examination order request (e.g., an order for an imaging service request) can include a study code and other order-related information (e.g., reason for the exam, order questions, and so forth). Conventionally, these imaging examination order requests are reviewed by an authorized party, which is hereinafter referred to as a “protocoler”, such as an imaging technologist, operator or other healthcare professional who will, based on the imaging examination order request, assign an imaging protocol for the medical or imaging examination procedure. An imaging protocol is a set of technical parameters, specific to an imaging system and contrast delivery.

### SUMMARY

The following presents a simplified summary of the specification in order to provide a basic understanding of some aspects of the specification. This summary is not an extensive overview of the specification. It is intended to neither identify key or critical elements of the specification, nor delineate any scope of the particular implementations of the specification or any scope of the claims. Its sole purpose is to present some concepts of the specification in a simplified form as a prelude to the more detailed description that is presented later.

In accordance with a non-limiting, example implementation, a system can include a protocoling component that receives a medical examination order request in a standardized input format. Based on a machine learning technique, the protocoling component can output a recommended protocol according to a standardized output format. The system can further comprise a generalizable component that can perform a mapping procedure. The mapping procedure can map site-specific data to the standardized input format and the standardized output format, wherein the site-specific data comprises information that is specific to an entity that provides the medical examination order request

The following description and the annexed drawings set forth certain illustrative aspects of the specification. These aspects are indicative, however, of but a few of the various ways in which the principles of the specification may be employed. Other advantages and novel features of the specification will become apparent from the following detailed description of the specification when considered in conjunction with the drawings.

### BRIEF DESCRIPTION OF THE DRAWINGS

Numerous aspects, implementations, objects and advantages of the present invention will be apparent upon consideration of the following detailed description, taken in conjunction with the accompanying drawings, in which like reference characters refer to like parts throughout, and in which:

FIG. 1 illustrates a high-level schematic flow diagram of an example medical examination procedure that leverages the disclosed techniques is presented in accordance with certain embodiments of this disclosure;

FIG. 2 depicts a schematic block diagram illustrating an example system that can facilitate generalizable machine learning medical protocol recommendations in accordance with certain embodiments of this disclosure;

FIG. 3 depicts an example schematic block diagram illustrating additional aspects or elements in connection with generalizable machine learning medical protocol recommendations in accordance with certain embodiments of this disclosure;

FIG. 4 depicts an example schematic flow diagram illustrating additional aspects or elements in connection with data interfacing in accordance with certain embodiments of this disclosure;

FIG. 5A depicts a diagram, illustrating an example order-level protocol in accordance with certain embodiments of this disclosure;

FIG. 5B illustrates a diagram representing a priority indication that can be embedded in HL7 messages in accordance with certain embodiments of this disclosure;

FIG. 6 depicts an example flow diagram, illustrating an example workflow for generalizable protocoling of an examination order request in accordance with certain embodiments of this disclosure;

FIG. 7 depicts an example flow diagram, illustrating an example workflow for generalizable protocoling based on a rules-based mapping procedure in accordance with certain embodiments of this disclosure;

FIG. 8 depicts an example flow diagram, illustrating an example workflow for generalizable protocoling based on a site-specific learning phase or procedure in accordance with certain embodiments of this disclosure;

FIG. 9 depicts a flow diagram of an example method for facilitating a generalizable machine learning medical protocol recommendation in accordance with certain embodiments of this disclosure;

FIG. 10 depicts a flow diagram of an example method for providing additional aspect or elements in connection with facilitating a generalizable machine learning medical protocol recommendation in accordance with certain embodiments of this disclosure;

FIG. 11 is a schematic block diagram illustrating a suitable operating environment in accordance with certain embodiments of this disclosure; and

FIG. 12 is a schematic block diagram of a sample computer communication environment in accordance with certain embodiments of this disclosure.

### DETAILED DESCRIPTION

Various aspects of this disclosure are now described with reference to the drawings, wherein like reference numerals are used to refer to like elements throughout. In the following description, for purposes of explanation, numerous specific details are set forth in order to provide a thorough understanding of one or more aspects. It should be understood, however, that certain aspects of this disclosure might be practiced without these specific details, or with other methods, components, materials, etc. In other instances, well-known structures and devices are shown in block diagram form to facilitate describing one or more aspects.

Upon reviewing the order for an imaging service request, a protocoler, such as an imaging technologist, operator or other healthcare professional will typically assign an order protocol (e.g., a radiology protocol) to be used. Thereafter, the same or a different protocoler can assign a machine-level protocol that is specific to the device (e.g., a CT device) that will effectuate the order protocol. It is appreciated that the order protocol is distinct from the machine-level protocol, but the disclosed techniques can be implemented for both. It is further understood that, individually, the required protocoling is not particularly time-intensive, but in the aggregate, this aspect of the medical doctor's or radiologist's (or other suitable healthcare professional) duties can become onerous and repetitive without significant focus, and it is not uncommon that mistakes occur or that certain procedures or imaging studies will need to be repeated. Moreover, an order for an imaging service request can be utilized to schedule and perform imaging services for a patient.

Systems and techniques detailed herein are directed to automating this protocoling process, which can mitigate human-error as well as free up the protocoler to focus more intently on other tasks. In the context of this disclosure, the medical protocoling detailed is specific to radiology, but it is understood that such is intended to be a representative example, and other medical fields or domains can benefit from the disclosed techniques in a similar manner.

One challenge associated with automating protocoling processes is that machine learning techniques typically expect standardized inputs in order to generate standardized outputs. Training the model generally requires examining large sets of data that takes on the context of a specific entity (e.g., a specific hospital). Individual hospitals or other relevant entities may rely on different policies and practices, may rely on different terminology, and might also classify data differently, resulting in different characteristics such as using different age groups, different order types such as inpatient, outpatient, emergency, and so on. Thus, a machine learning model arduously trained for one hospital is not likely to be useful or applicable to a different hospital, instead requiring the training to be repeated for each different entity in order to be useful.

Hence, a model that is generalizable would be a welcome advance in the industry. In accordance with techniques detailed herein, a machine learning model can be trained according to standardized inputs and outputs, which can rely on a significant amount of data and training. However, rather than repeating this process for every different entity, only a small fraction of the original data and training can be used to allow the standardized outputs to be transformed into site-specific outputs suitable for different entities.

Furthermore, the disclosed techniques can rely on a decision support aspect that can identify when the model fails to produce a reliable recommendation and/or can distinguish between routine protocoling and non-routine protocoling, or otherwise intelligently identify scenarios that lend themselves to notifying a protocoler. As those cases will likely be rare, the protocoler will be more likely to carefully examine the associated order requests, potentially mitigating conventional errors.

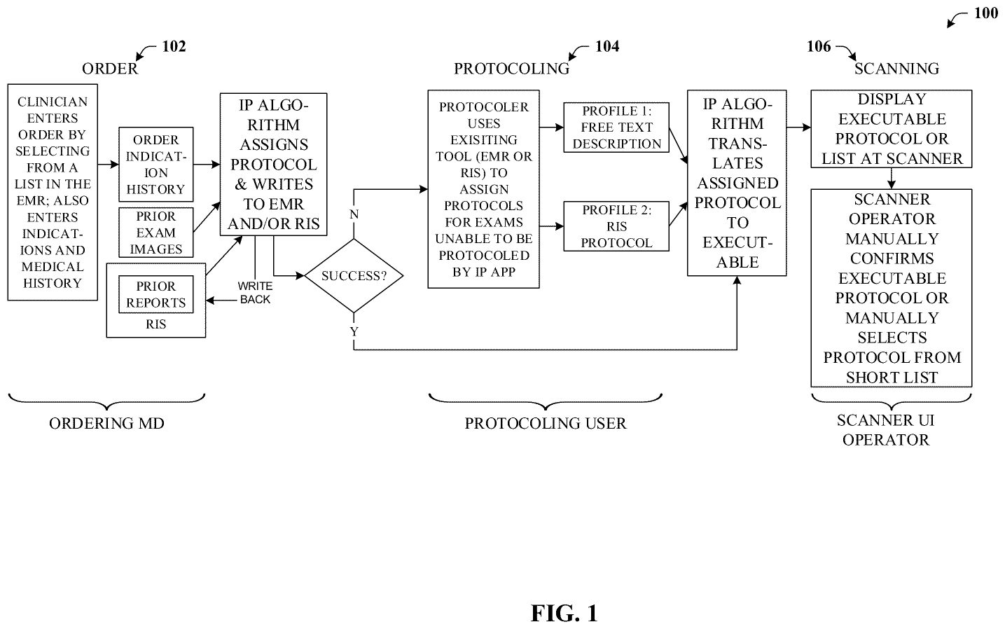

Referring initially to FIG. 1, a schematic flow diagram 100 of an example medical examination procedure that leverages the disclosed techniques is presented in accordance with certain embodiments of this disclosure. In this example, the flow is categorized at a high level into three distinct categories, denoted order 102, typically handled by an ordering medical doctor or other clinician; protocoling 104, typically overseen by a protocoler; and scanning 106, typically handled by a radiologist, imaging technologist, operator or other healthcare professional. As this example is intended to be high-level, additional details are discussed in connection with subsequent drawings.

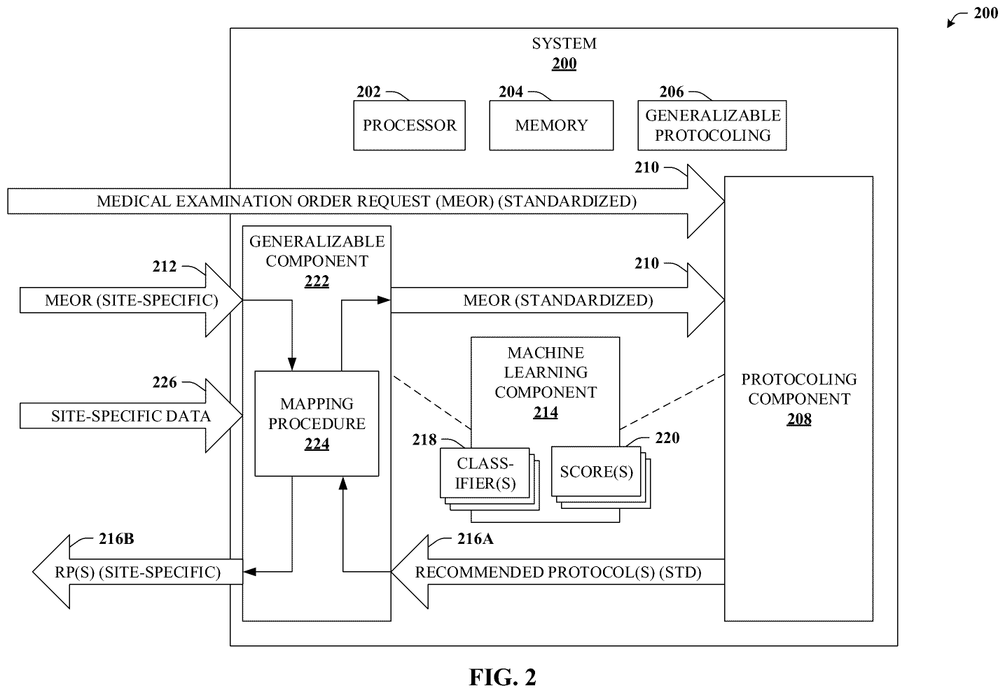

Within the order 102 category, a clinician fills out an examination order request, which is typically accomplished by selecting from a list in the electronic medical record (EMR). The clinician can further enter patient indications and medical history information as well as prior examination procedure images or other information and prior reports, which can be accessed from a radiology information system (RIS) or another suitable system. This examination order request can be received by the intelligent protocoling (IP) system, which is further detailed in connection with FIG. 2. In some embodiments, the examination order request can be received in a Health Level 7 (HL7) format (e.g., version 1, version 2.x, version 3, . . . ), a Fast Healthcare Interoperability Resources (FHIR) format, a continuity of care document (CCD) format or another suitable format. The examination order request can include a study code and order-related information such as a reason for the exam, order questions, and so forth, and other clinical information, e.g., from an electronic health record (EHR) or other hospital information system (HIS), which may rely on FHIR, cross-community access (XCA), cross-enterprise document sharing (XDS), technology.

Via machine learning techniques detailed infra, the IP system can assign a recommended protocol to the examination order request. Single or multiple recommendations can be determined, either of which can be verified by the protocoler. Regardless, the final selected protocol can be written back to the RIS or HIS.

In more detail, as illustrated by the protocoling 104 category, if the machine learning techniques do not result in a viable recommendation, a protocoler can be notified, for example, via a user interface (UI) that is part of the IP system. Inputs to the UI can be a free text description or in RIS protocol. In either case, or if a recommendation is made, the IP system can translate the assigned/recommended protocol to a machine-level protocol.

As shown under the scanning 106 category, when the patient arrives at the scanner, the translated, executable protocol(s) can be displayed at the scanner (e.g., CT scanner, . . . ). The scanner operator can manually confirm the executable protocol or manually select from a short list of executable protocols.

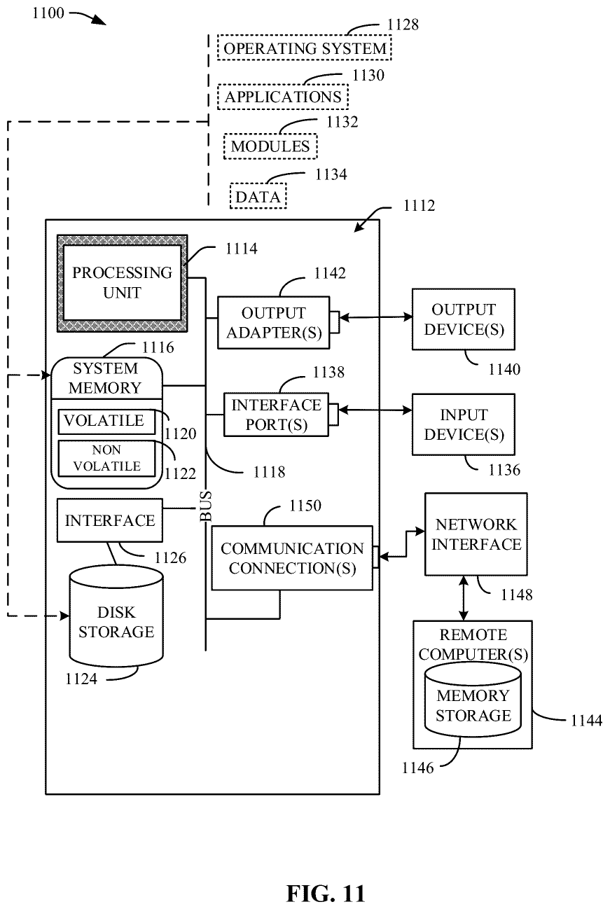

Referring now to FIG. 2, a schematic block diagram is depicted illustrating an example system 200 that can facilitate generalizable machine learning medical protocol recommendations in accordance with certain embodiments of this disclosure. For example, system 200 can provide generalizable protocoling 206 that can be utilized regardless of site-specific distinctions between hospitals or other entities or institutions. System 200 can comprise a processor 202 that can be specifically configured to provide generalizable protocoling 206. System 200 can also comprise memory 204 that stores executable instructions that, when executed by processor 202, can facilitate performance of operations. Processor 202 can be a hardware processor having structural elements known to exist in connection with processing units or circuits, with various operations of processor 202 being represented by functional elements shown in the drawings herein that can require special-purpose instructions, for example stored in memory 204 and/or generalizable protocoling 206 component or circuit. Along with these special-purpose instructions, processor 202 and/or device 200 can be a special-purpose device. Further examples of the memory 204 and processor 202 can be found with reference to FIG. 11. It is to be appreciated that system 200 or computer 1112 can represent a server device or a client device and can be used in connection with implementing one or more of the systems, devices, or components shown and described in connection with FIG. 2 and other figures disclosed herein.

System 200 can further comprise protocoling component 208. Protocoling component 208 can receive a medical examination order request (MEOR) 210 in a standardized input format. As noted above, when a clinician fills out a MEOR, such is commonly done according to standardized formats such as HL7, FHIR, or the like. However, in the event that the MEOR is not standardized (illustrated as site-specific MEOR 212), then such can be translated and/or mapped into MEOR 210 in a manner similar to that described below in connection with system outputs. In other words, while this disclosure focuses extensively on mapping machine learning outputs to site-specific outputs to enable the generalizable application, it is appreciated that such mapping can be implemented on the input side as well.

In either case, protocoling component 208 receives MEOR 210 that is in a standardized format. Based on machine learning techniques provided by machine learning component 214, protocoling component 208 can output a recommended protocol 216A that is formatted according to a standardized output format.

Machine learning component 214 can include one or more classifiers 218 that can be trained. Classifiers 218 can be any suitable machine learning classifier such as, for example, a naïve Bayesian classifier or the like. Once trained, the machine learning techniques can determine scores 220, also referred to as confidence scores 220, which can indicate a confidence of classifier 218 or other machine learning elements being correct or suitable. For example, recommended protocol 216A can be selected from among other potential protocols based on a values of associated confidence scores 220. For instance, recommended protocol 216A can be the protocol having a highest confidence score.

In some embodiments, recommended protocol 216A can comprise multiple recommended protocols 216A. For example, the top n protocols, where n is some integer greater than one. In some embodiments, n can be static, e.g., the top two or three protocols as determined by associated confidence scores 220. In that case, n can be determined in advanced. In other embodiments, n can be dynamic or variable such that that the number of recommended protocols 216A output can be a function of confidence score 220, such as only recommend protocols with associated confidence scores 220 that are above a define threshold. In that case, the number of recommended protocols 216A that are output can be limited only to those protocols with higher confidence scores 220. In some embodiments, n can be a combination of these two techniques, e.g., output the top three, but only if the associated scores 220 are above a defined value.

As noted, recommended protocol 216A is in a standardized format based on the training set and may not be consistent with site-specific data and policies. In one sense, protocoling component 208 can represent a standardized, pre-trained model. However, in addition to protocoling component 208, system 200 can further comprise generalizable component 222 that can effectuate a generalizing capability so that outputs of the standardized model, that are typically not appropriate for a specific site, can be transformed to a site-specific output that can be appropriate for a given site.

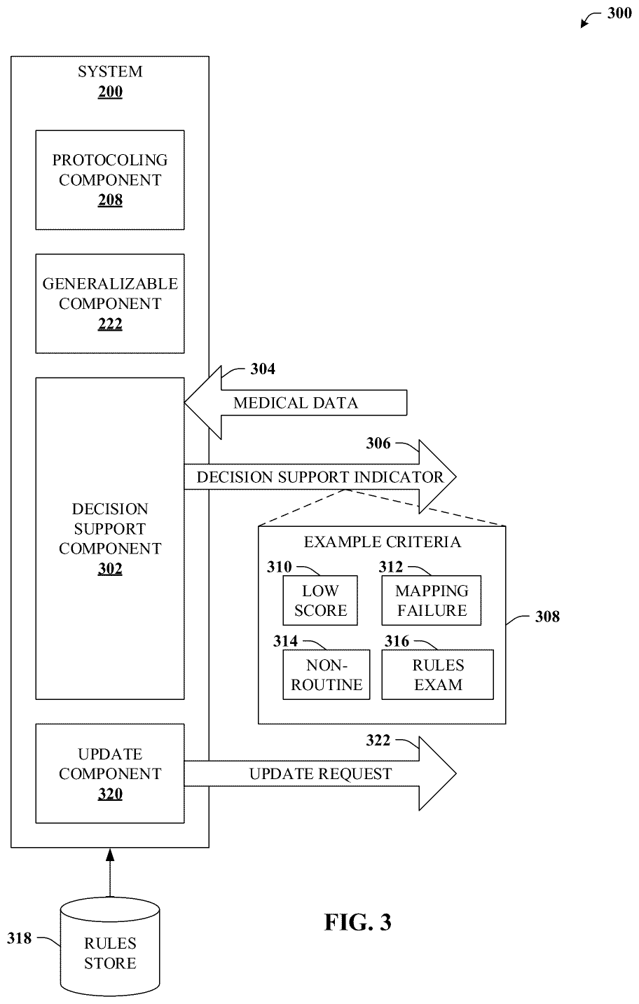

In that regard, generalizable component 222 can perform mapping procedure 224 that can map site-specific data 226 to the standardized input format and the standardized output format and vice versa. Site-specific data 226 can comprise information that is specific to an entity that provides MEOR 210, 212 and can be significantly less than what is utilized to train the models of protocoling component 208. In other words, while recommended protocol 216A makes sense in the standardized context, recommended protocol 216B, output by the generalizable component 222 can be specific to the site that requests the information. Additional detail regarding techniques associated with mapping procedure 224 and/or generalizable component 222 (e.g., based on a rules-based procedure) can be found with reference to FIG. 3.

Turning now to FIG. 3, an example schematic block diagram is depicted illustrating additional aspects or elements in connection with generalizable machine learning medical protocol recommendations in accordance with certain embodiments of this disclosure. For example, in addition to protocoling component 208 and generalizable component 222 detailed in connection with FIG. 2, system 200 can further comprise decision support component 302.

Decision support component 302 can determine whether or not support from a protocoler is to be requested, e.g., by notifying the protocoler. Such can be accomplished by examining medical data 304 and, in response, outputting decision support indicator 306 based on criteria 308. Medical data 304 can include any data available to other components of system 200 such as MEOR 210, 212 and recommended protocol 216A, 216B and so forth. As a simple example, decision support indicator 306 can indicate that a protocoler is to be notified in response to recommended protocols 216A, 216B indicating multiple protocols. Multiple protocols can be output along with associated confidence scores 220, or a suitable representation, yet, ultimately, a protocoler might be requested to choose between the multiple recommendations, particularly in cases where respective scores 220 are both high and close.

Other examples of criteria 308 can include an associated confidence score 220 being too low, as illustrated by reference numeral 310, a mapping failure 312 or a determination that the MEOR 210 relates to a non-routine examination. In more detail, if a confidence score 220 for an associated MEOR 210 is below a defined threshold, then such can trigger decision support indicator 306 to notify the protocoler. If generalizable component 222 cannot adequately map (e.g., via mapping procedure 224) recommended protocol 216A to recommended protocol 216B, then such might also trigger decision support indicator 306 to notify the protocoler. In some embodiments, decision support component 302 can identify non-routine 314 analysis. For example, if protocoling component 208 fails to generate recommended protocol 216A, then such can be considered non-routine 314.

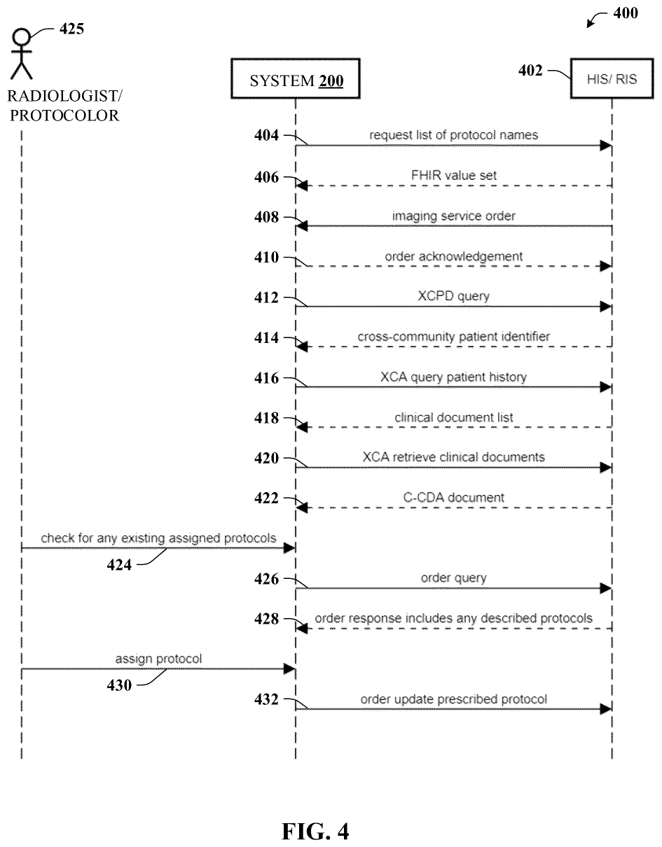

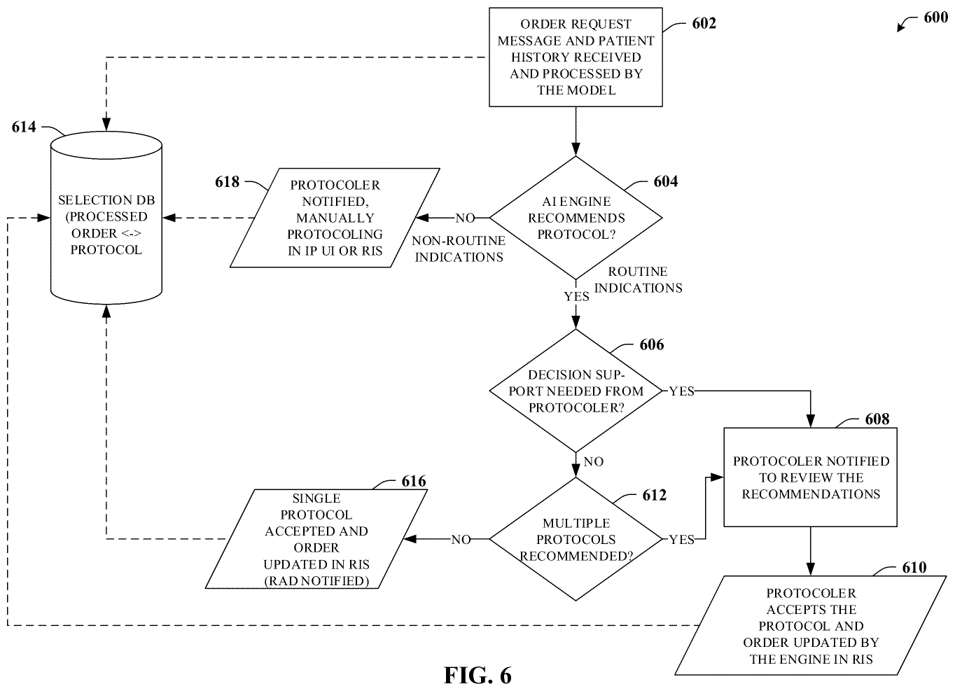

In addition, medical data 304 can include patient records such as an EHR or other information, which is further detailed in connection with FIG. 4. In that case, criteria 308 that decision support component 302 utilizes to determine decision support indicator 306 can include a rules-based examination 316 of the EHR or other information. For example, based on rules-based examination 316 of the EHR, consider it is determined that the patient is allergic to a particular contrast associated with a particular medical procedure. Such can be a factor in decision support indicator 306 indicting that the protocoler is to be notified. Additional detail regarding decision support and other aspects or elements can be found with reference to FIG. 6.

Rules-based examination 316 can be performed based on a defined set of rules, which can be stored in rules store 318. Rules store 318 can further store rules that, as part of a rules-based procedure, are employed to associate site-specific protocols included in site-specific data 226 to standardized output formats (or input formats). In other words, mapping procedure 224 can use this set of rules to transform recommended protocol 216A into recommended protocol 216B, whereas decision support component 302 can utilize a different set of defined rules.

System 200 can further comprise update component 320 that can periodically transmit update request 322. Update request 322 can request updates to site-specific data 226, which is further detailed in connection with FIG. 4.

FIGS. 4-8 are intended to provide addition disclosure relating to the techniques detailed herein. Generally speaking, the discussion of FIGS. 4-8 can be divided into four sections, namely, a protocol bundle transaction section, an order initiation section, a order-level protocoling section, and a machine-level protocoling section.

The protocol bundle transaction section generally describes a mechanism by which the intelligent protocoling (IP) engine (e.g., system 200) can request the list of all site-specific medical (e.g., radiology) protocols from the RIS and store them in a data store. In addition, example approaches are detailed to keep the list of protocols in the IP engine updated and in synch with the RIS protocols.

The order initiation section describes a mechanism that is utilized by the IP engine to obtain the examination order request (e.g., MEOR 210, 212) once it is completed by the medical doctor or clinician and submitted to the EHR. These order requests can be filtered and/or prioritized based on the content of the order request.

The order-level protocoling (also referred to herein as protocoler-level protocoling to distinguish between machine-level protocoling for a scanner or other device) section describes a human-machine system where the IP engine provides single or multiple recommendations (e.g., recommended protocol 216A, 216B) for a subset of the examination orders (e.g., MEOR 210) while providing no recommendation for other orders (e.g., low score 310, mapping failure 312, non-routine 314, . . . ). If there is no recommendation or a recommendation deemed unsatisfactory (e.g., low score 310), decision support elements can be initiated to notify a protocoler to perform the protocoling manually. Such can be accomplished via a UI provided by the IP engine or in the RIS. The final selected protocol can then be recorded back into the database of the RIS. The decision of the IP engine to provide recommendations or not can be based on the output of a machine learning classifier (e.g., classifier 218).

The machine-level protocoling (also referred to herein as scanner-level protocoling) section details a subsequent step in the protocoling process in which the IP engine recommends the imaging protocols based on the selected order-level protocol that was recommended (e.g., recommended protocol 216A) or otherwise written back to the RIS. In some embodiments, the imaging protocols can relate to a set of technical parameters that are specific to the particular imaging equipment being used and/or contrast delivery.

With reference now to FIG. 4, an example schematic flow diagram 400 is depicted illustrating additional aspects or elements in connection with data interfacing in accordance with certain embodiments of this disclosure. With regard to protocol bundle transactions, it is appreciated that the IP engine and/or system 200 relies on access to the site-specific list of medical protocols (e.g., radiology protocols), which can be obtained or periodically updated from the relevant HIS/RIS, denoted by reference numeral 402. At reference numeral 404 system 200 can request the list of protocol names. At reference numeral 406, system 200 can, in response, receive this list, which can be formatted according to a FHIR value set. This information can be utilized by system 200 to provide recommended protocols 216A, 216B after applying machine learning techniques on the information provided by response 406 as well as information from MEOR 210 and patient history information (e.g., EHR).

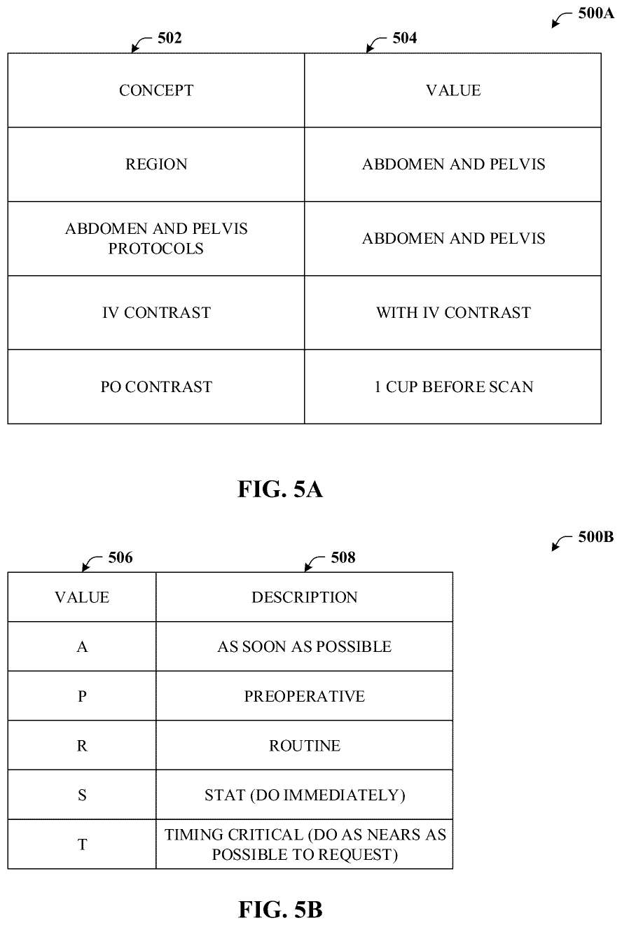

Sites that have a protocoling module in their RIS store these site-specific protocols electronically in the RIS. Thus, at 404, system 200 can request a list of order-level protocols. The complete list of order-level protocols can then be obtained via 406. At 408, an imaging service order can be received, and at 410, that order can be acknowledged. The first two query and response procedures (e.g., reference numerals 404-410) can demonstrate a mechanism to receive a list of order-level protocols from an institution's (e.g., hospital) radiology (or other) protocoling module. The FHIR query can be performed based on the modality and anatomy (or region) such as, e.g., “Head”, “Abdomen”, and so forth. It should be appreciated that some sites may store their order-level protocols indexed by the modality and region as illustrated in FIG. 5A.

FIG. 5A depicts a diagram 500A, illustrating an example order-level protocol in accordance with certain embodiments of this disclosure. An order-level protocol can comprise a concept 502 and a value 504. For instance, concept 502 can indicate an anatomical region, and can also include other concepts 502 such as contrast and the like. As illustrated, each concept 502 can have an associated value 504. The value 504 can be formatted as text, as shown here, but can be in other formats as well in other embodiments.

Still referring to FIG. 4, it is appreciated that sites that do not have a protocoling module in their RIS or HIS can rely on free-text order-level protocols to instruct the technologist during the scanning. It is noted that the order-level protocoling section below describes example mechanisms that can be utilized by system 200 to build a list of order-level protocols for such sites. Furthermore, it is noted that individual sites may also update their lists of order-level protocols and these updates should be synchronized with the information on-hand for system 200. Update component 320 (of FIG. 3) can perform updates in various ways. One way is to conform to a FHIR subscription. Another technique is to periodically send update request 322, requesting protocol value sets as illustrated at 404. Such can be done based upon a determined or anticipated frequency of updates such as, e.g., nightly updates.

Continuing review of flow diagram 400, at 412, system 200 can transmit an XCPD query. At 414, HIS/RIS 402 can respond with a cross-community patient identifier, which can be used at 416 to request XCA patient history. At 418, HIS/RIS 402 can respond with a clinical document list. At 420, these clinical documents can be requested, and at 422, that query can be satisfied, e.g., by a consolidated-clinical document architecture (C-CDA) document.

At this point, system 200, will generally be able to make a recommendation. In that regard, steps illustrated by reference numerals 404-410 can be performed in advance. Upon receiving MEOR 210, steps indicated by reference numeral 412-422 can be initiated. At reference numeral 424, a check can be performed for any existing assigned protocols, e.g., those already protocoled by a protocoler 425. If a given MEOR 210 is already protocoled, system 200 need not do so, however, in some cases, such can be performed and the output used for training, e.g., by comparing the protocol generated by protocoler 425 to recommended protocol 216A, 216B.

At 426, the order can be query and, in response, at 428, an order response can be received. This order response can include any described protocols. At 430, protocoler 425 can assign the protocol, which, ideally, will be accepting recommended protocol 216A, 216B. At 432, the order is updated according to the prescribed protocol.

Regarding the order initiation section, it is appreciated that in a radiology center (or other medical site) the workflow is typically initiated with a referring physician entering an order in the EHR describing the reason for the examination and a study code. The order is then communicated to the hospital RIS typically via an HL7 v2 message. An example of an HL7 v2 message is provided below:
MSH|\hat{ }˜\&|CompanyName|SHC|∥20210408085800∥ORM\hat{ }O01|5878|T|2.5
PID|∥10013431∥TESTING\hat{ }RESARCH∥19610101|M
PV1∥O|BLAKEWCT\hat{ }\hat{ }\hat{ }SHC BLAKE WILBUR\hat{ }\hat{ }\hat{ }\hat{ }\hat{ }\hat{ }\hat{ }
ORC|NW|300065032\hat{ }EPC|TST17197549∥∥∥∥∥S052\hat{ }\hat{ }\hat{ }110999\hat{ }\hat{ }\hat{ }\hat{ }\hat{ }DIAGNOSTIC RADIOLOGY SPLTY∥∥|BLAKEWCT\hat{ }\hat{ }\hat{ }SHC BLAKE WILBUR
OBR∥300065032\hat{ }EPC|TST17197549|IMGCT0047\hat{ }CT HEAD ANGIOGRAPHY W AND WO IV CONTRAST\hat{ }|CompanyName∥20210407∥∥|(408) 851-4010∥These are order comments entered while placing the order˜∥HEAD\hat{ }CT NEURO|054827\hat{ }SMITH\hat{ }ALLISON\hat{ }J\hat{ }\hat{ }\hat{ }\hat{ }\hat{ }SHC\hat{ }\hat{ }\hat{ }\hat{ }MSPV∥TST17197549∥∥∥CT|NW∥\hat{ }\hat{ }\hat{ }\hat{ }\hat{ }R∥∥∥∥|20210408090000
OBX|1|ST|IRAD50\hat{ }Iodine contrast allergy?|1|Unknown∥∥∥F∥20210407083203∥∥∥∥∥∥∥|QST∥|
OBX|2|ST|IRAD96\hat{ }Want immediate contact?|1|No∥∥|F|∥20210407083203∥∥∥∥∥∥∥|QST|∥
OBX|3|ST|IRAD97\hat{ }Exam modification OK?|1|Yes∥∥∥|F|∥20210407083203∥∥∥∥∥∥∥|QST|∥
OBX|4|ST|IRAD31 \hat{ }Reason∥Head trauma, mod-severe (Ped 0-18y)

In this HL7 v2 message, the OBX-4 segment includes the “reason for exam” (e.g., Head trauma, mod-severe (Ped 0-18y)) and the OBR-4 segment includes the “study code” being requested. There are other OBX segments that describe other order related questionnaire.

The above example represents a standard HL7 v2 message, which can be directed to system 200 as well. Such can initiate an action in which system 200 can query the C-CDA in the hospital EHR (e.g., HIS/RIS 402), as illustrated at reference numeral 420. As indicated, the workflow for initiating the patient history can be XCA, illustrated at 416. However, it is understood that patient history can be retrieved by other mechanisms as well, such as an HL7 v2, HL7 FHIR, or an XDS query.

In addition, an HL7 message has the capability to reflect priority of the imaging order being requested. Turning now to FIG. 5B, diagram 500B is presented. Diagram 500B represents a priority indication that can be embedded in HL7 messages in accordance with certain embodiments of this disclosure. Diagram 500B illustrates various priority values 506 and associated descriptions 508. These values, which can be present in an HL7 v2 message can be utilized by system 200 to prioritize recommendations or other processing.

In addition to the above-mentioned priority, system 200 can further employ different models or techniques for different study codes. For example, consider the following three examples, labeled a)-c):
a) CT ABDOMEN PELVIS—Region (ABD+PELVIS), Modality Modifier (UNKNOWN)
b) CT SACROPLASTY BILATERAL—Region (Pelvis), Modality Modifier (UNKNOWN), It is not a diagnostic imaging order, it is a procedure order
c) CT ANGIO ABDOMEN PELVIS—Region (ABD+Pelvis), Modality Modifier (ANGIO)

The study code in a) may employ a Body CT specific model while c) may employ an angio-specific model or avascular-specific model. System 200 can classify (e.g., via classifiers 218) the study codes into, e.g., REGION and MODALITY_MODIFIER and determine whether the study code is for body CT or angio.

It is noted that the same approach may not operate to differentiate study codes a) and b). In this case, a) is a diagnostic imaging order while b) is an image guided procedure. However, both of these examples have the same REGION and MODALITY_MODIFIER. The RADLEX playbook does define such image guided procedures as belonging to a different modality called Radiology Procedure, which can be reflected in the study codes if the organization codes are mapped to the playbook.

Regarding the order-level protocoling section, FIGS. 6-8 can be reviewed. With reference now to FIG. 6, flow diagram 600 is depicted. Flow diagram 600 illustrates an example workflow for generalizable protocoling of an examination order request in accordance with certain embodiments of this disclosure. It is appreciated that workflow 600 leverages concepts introduced above in connection with system 200 and can demonstrate additional aspects or elements. Workflow 600 can begin after system 200 has received MEOR 210 and associated patient history as described above, which is illustrated here at reference numeral 602.

Leveraging machine learning techniques, system 200 can operate to recommend one or multiple protocols (e.g., recommended protocol(s) 216A, 216B), potentially along with rankings associated to each one based on confidence scores 220 assigned to each particular protocol, which occurs at reference numeral 604. If the recommendation fails, system 200 may not provide any recommendation depending on the output of the particular machine learning classifier (e.g., classifier 218). In that regard, a multi-class machine learning classifier 218 can be trained with one of the classes representing all non-routine indications. The decision of whether or not an indication is routine can be trained based on the frequency of the occurrence or another suitable factor. Table I below shows an example of a routine and a non-routine indication as determined by an example classifier 218.

TABLE I

| Order | Indication | Is Routine ? |
|---|---|---|
| LUQ pain after | Left upper quadrant | Y |
| eating × 3 weeks | pain | |
| h/o alpha thalassemia, | Thalassemia, alpha | N |
| gallstones, r/o | | |
| hepatosplenomegaly | | |

Typically, orders that are determined to be non-routine may not be protocoled by system 200. In some embodiments, when system 200 fails to make a recommendation, such can be an indication that the order is non-routine. In such cases, the flow can proceed to 618. The protocoler can be notified and the order can be protocoled by the protocoler in a UI provided by the system 200 or the RIS.

Otherwise, system 200 can rank the protocols based on confidence scores 220, and the flow proceeds to 606, where a decision support examination is made to determine whether it is recommended that the protocoler review the recommendation. Such can be implemented based on decision support indicator 306, for example. If so, the flow proceeds to 608, where the protocoler is notified to review the recommendation(s). At 610, the protocoler assigns the protocol, whether a recommended one or another, and the associated order is updated by system 200 in the RIS or other appropriate data store. If not, the flow proceeds to 612.

System 200 can recommend a single or multiple protocols by applying a numerical criterion on the confidence scores 220 associated with the protocols. If a single protocol is recommended, the flow can proceed to 616, where the single protocol can be accepted and the order updated in the RIS. In some cases the protocoler can be notified here as well. On the other hand, if multiple protocols are recommended, the flow can proceed to 608 for protocoler review, as detailed above. The protocoler can then select the final protocol, which can be written to the RIS or other associated data store, as indicated at 614.

As discussed previously at FIG. 3, and illustrated here at 606, system 200 can implement decision support techniques. For example, system 200 can incorporate a rules-based decision support system to handle situations in which, e.g., a patient has poor renal function, is on dialysis, is allergic to contrast, and so forth. Generally speaking, the policies to give contrast is institution-specific, changes over time, and can also depend on the medical condition. The estimated glomerular filtration rate (eGFR) level, active problems such as dialysis, and lists of allergens and their corresponding reactions can be present in an EHR associated with the patient.

In that regard, an example of patient allergies and other information is indicated below in Table II.

TABLE II

| Allergen | Reactions |
|---|---|
| Ephinephrin | Sometimes I pass out |
| Mussels | NAN |
| Iodine and Iodine | Patient states when she has Iodine for |
| containing products | procedures such as CT/MRI she |
| | begins to have Tachycardia and must |
| | be taken to the ER Cardiac arrest per |
| | daughter |
| Codeine | Pt states she passes out |

As one example, the decision support system (e.g., decision support component 302) can then parse through the EHR data and trigger the engine to notify the protocoler to review the recommended protocols and take select appropriate protocol based on the medical need and patient's history. Regardless, the final selected protocol can then be written back to the RIS, e.g., via HL7 v2 order update message, in addition to any free text notes from the protocoler, which is illustrated at 614. Further, should there be a deviation between the recommended and selected protocol, such can be used to train and improve the model, since both the recommended and selected protocol can be save to a data store.

It is appreciated that flow 600 represents implementations in which system 200 has been trained on the order-level protocols that are site-specific. However, order-level protocols are not standardized across sites and usage of protocols can vary significantly. As such, a site-specific, generalizable model, as proposed herein, can address many associated deployment challenges. In that regard, the disclosed techniques can rely in part on a pre-trained model that has been trained using data potentially having different characteristics such as different age groups, different order types such as inpatient, outpatient, emergency, and so forth. Thereafter, as detailed above in connection with FIGS. 2 and 3, generalizable component 222 can utilize various different approaches to implementing a site-specific classifier engine.

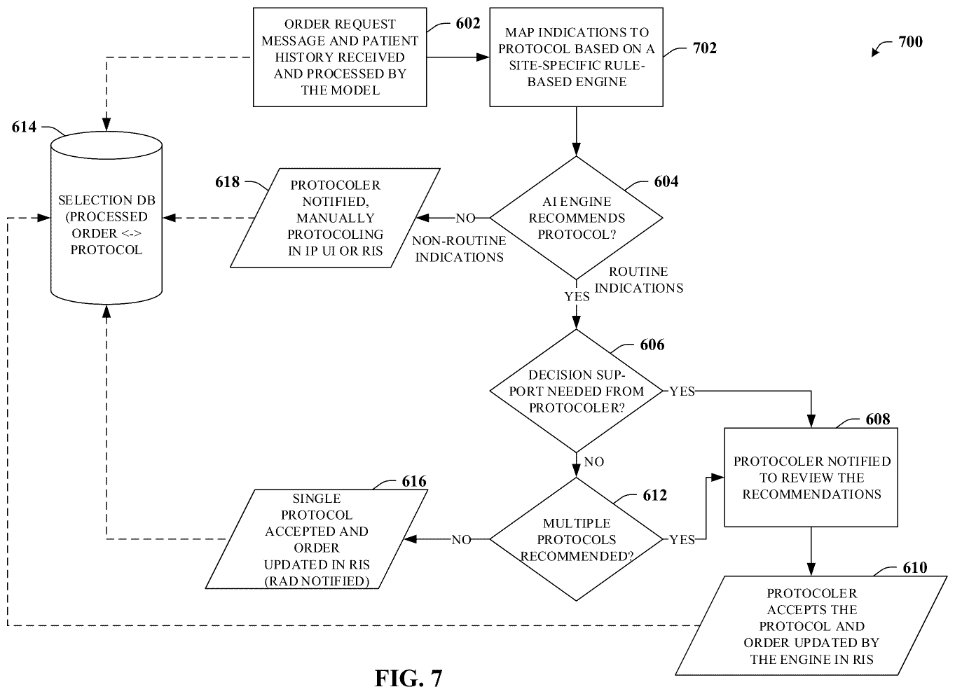

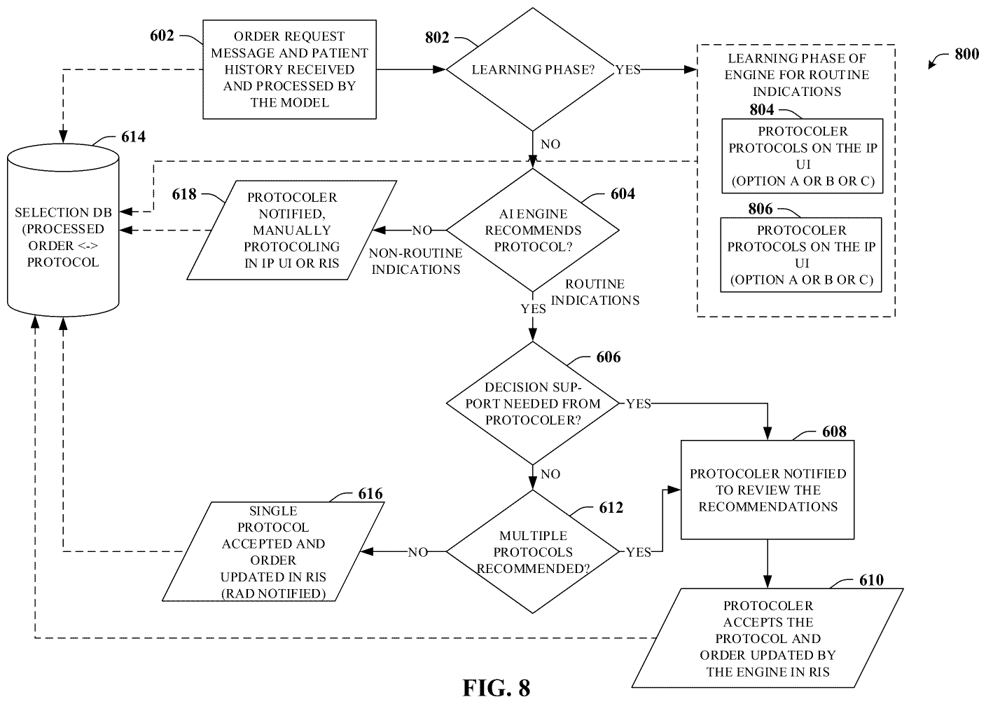

A first approach is illustrated in connection with FIG. 7 that details a rules-based mapping procedure, while a second approach is illustrated in connection with FIG. 8 that details a site-specific learning phase. In either case, these below discussion can be applicable to generalizable component 222, as detailed herein.

Referring now to FIG. 7, flow diagram 700 is depicted. Flow diagram 700 illustrates an example workflow for generalizable protocoling based on a rules-based mapping procedure in accordance with certain embodiments of this disclosure. Flow 700 can be substantially similar to flow 600 described in connection with FIG. 6. In addition to what was described in flow 600, flow diagram 700 can, as illustrated at reference numeral 702, rely on a rules-based mapping (e.g., see mapping procedure 224 of FIG. 2 and rules store 318 of FIG. 3 and associated text). This rules-based mapping can map (potentially standardized) clinical indications to site-specific protocols. The rules-based mapping can be implemented at every site and can rely on updates (e.g., update request 322 of FIG. 3) as new indications are added to the site-specific store or old indications are modified. On advantage of the rules-based mapping is that no additional learning phase is relied on compared to the second approach described below. Rather, the workflow can be up and running without significant training.

With reference now to FIG. 8, flow diagram 800 is depicted. Flow diagram 800 illustrates an example workflow for generalizable protocoling based on a site-specific learning phase or procedure in accordance with certain embodiments of this disclosure. Once more, flow 800 is substantially similar to flow 600 described in connection with FIG. 6. A distinction in this case is learning phase 802. In addition to deployment of a pre-trained model (e.g., protocoling component 208), once implemented at the site, such can be followed by site-specific learning phase 802 that can differ at each individual site. During learning phase 802, a classifier 218 can be trained to classify into site-specific protocols. As one example, learning phase 802 can be trained on data from different age groups, order types, and so forth according to the particular site. The model can thus learn site-specific protocols during learning phase 802.

During learning phase 802, system 200 will not typically provide the protocoler with recommendations. Rather, learning phase 802 can rely on selections made by the protocoler to learn. The protocoler can be provided with a UI where the site-specific radiology or other order-level protocols are displayed and the final selection can be made. The order-level protocol is then written back to the RIS using an HL7 v2 message, for example. In that regard, consider the following example implementation options, labeled as options a)-c):
a) Protocoler is presented with a user interface with options as shown in FIG. 5A, of an example protocol. The UI can also provide a library of options after initial manual configuration at the site (e.g., for sites that do not have a protocoling engine in their RIS) or automatic configuration (e.g., for sites that do have a protocoling engine in their RIS).
   I. The manual configuration can be relied upon when the site uses free text to describe the site-specific order-level protocols. The manual configuration may involve structuring their free text protocols into a format similar to that described in FIG. 5A.
   II. Automatic configuration can be performed by doing protocol bundle transaction first, e.g., using FHIR API, as described above in the protocol bundle transaction section, and then setting up the UI according to the data model used to describe the order-level protocol at the site. One such data model is described in FIG. 5A.
b) Protocoler is presented with a user interface with options, like the example protocol shown in 5A, and the protocoler can write free text describing those options. System 200 can then map the text to a standardized format. This option can be relied upon when manual configuration in step a) is not possible or infeasible at a site.
c) System 200 can receive the scanner protocols from the site and, for example, can map those site-specific protocols into the above 4 concepts defined in FIG. 5A. The options can be provided as a library of options. This option can be relied upon if manual configuration in step a) is not feasible at a site.

Of the options a)-c), option a) is generally the simplest and most robust approach for the machine model development for sites that rely on free text notes for their order-level protocols, and may be preferable in terms of ease of use for the protocoler. Option b) can rely on robust text matching technique that might require calculating metrics such as cosine distance between the entered text and all clusters to perform an unsupervised clustering. The clustering algorithm can then be supplemented by the protocoler's judgement. Option c) may involve developing a robust classifier for the scanner protocols to classify them into the format described in FIG. 5A. Such might also rely on collecting a robust set of annotations and training data that will be specific to each site.

Regarding the machine-level protocoling section, it is appreciated that upon completion of the order-level protocoling, additional steps can be provided. For instance, after the order-level protocoling, the patient can be scheduled for an imaging exam after the order-level protocol has been approved.

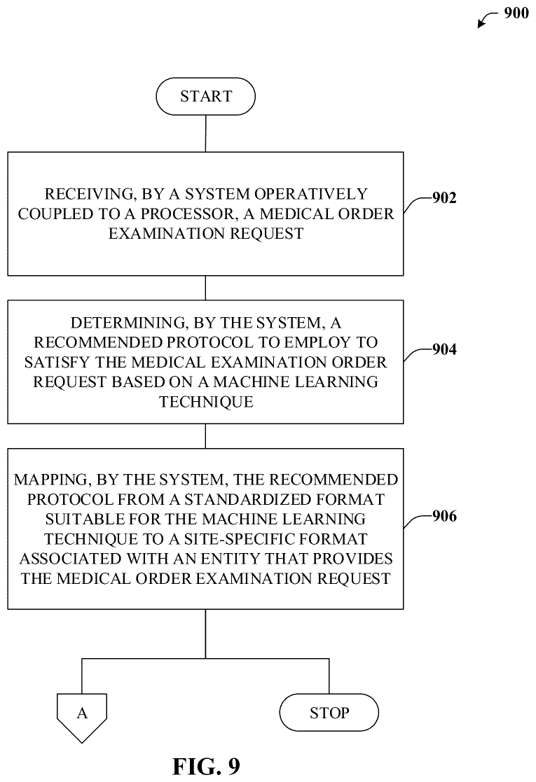

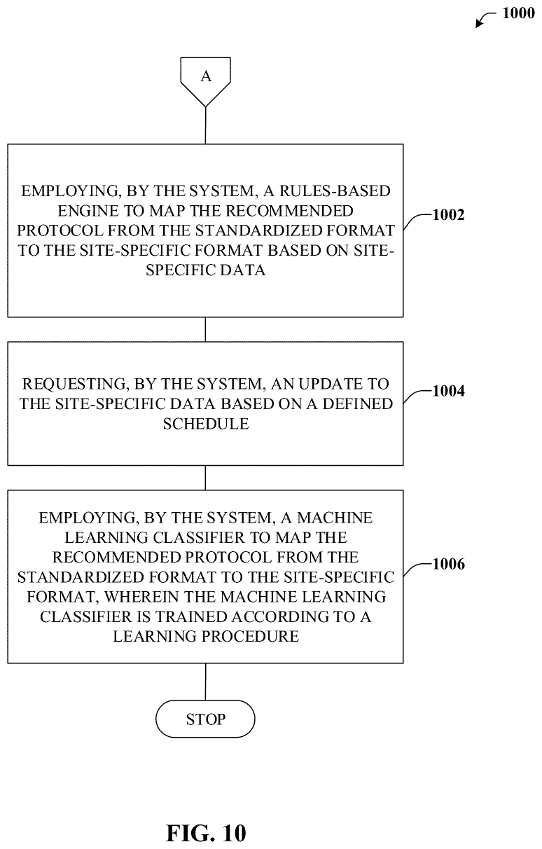

FIGS. 9 and 10 illustrate methodologies and/or flow diagrams in accordance with the disclosed subject matter. For simplicity of explanation, the methodologies are depicted and described as a series of acts. It is to be understood and appreciated that the subject innovation is not limited by the acts illustrated and/or by the order of acts, for example acts can occur in various orders and/or concurrently, and with other acts not presented and described herein. Furthermore, not all illustrated acts may be required to implement the methodologies in accordance with the disclosed subject matter. In addition, those skilled in the art will understand and appreciate that the methodologies could alternatively be represented as a series of interrelated states via a state diagram or events. Additionally, it should be further appreciated that the methodologies disclosed hereinafter and throughout this specification are capable of being stored on an article of manufacture to facilitate transporting and transferring such methodologies to computers. The term article of manufacture, as used herein, is intended to encompass a computer program accessible from any computer-readable device or storage media.

Referring to FIG. 9, there is illustrated a methodology 900 for facilitating a generalizable machine learning medical protocol recommendation in accordance with certain embodiments of this disclosure. For example, at reference numeral 902, a system operatively coupled to a processor can receive a medical order examination request.

At reference numeral 904, the system can determine a recommended protocol to employ to satisfy the medical examination order request based on a machine learning technique. At reference numeral 906, the system can map the recommended protocol from a standardized format suitable for the machine learning technique to a site-specific format associated with an entity that provides the medical order examination request. Method 900 can end or proceed to insert A, which is further detailed at FIG. 10.

Turning now to FIG. 10, there illustrated is a methodology 1000 for providing additional aspect or elements in connection with facilitating a generalizable machine learning medical protocol recommendation in accordance with certain embodiments of this disclosure. At reference numeral 1002, the system can employ a rules-based engine to map the recommended protocol from the standardized format to the site-specific format. This mapping can be based on site-specific data.

At reference numeral 1004, the system can request an update to the site-specific data based on a defined schedule. The schedule can be recurring based on appropriate times (e.g., nightly) or event-based and triggered in response to an occurrence such as changes at the site.

At reference numeral 1006, the system can employ a machine learning classifier to map the recommended protocol from the standardized format to the site-specific format, wherein the machine learning classifier is trained according to a learning procedure.

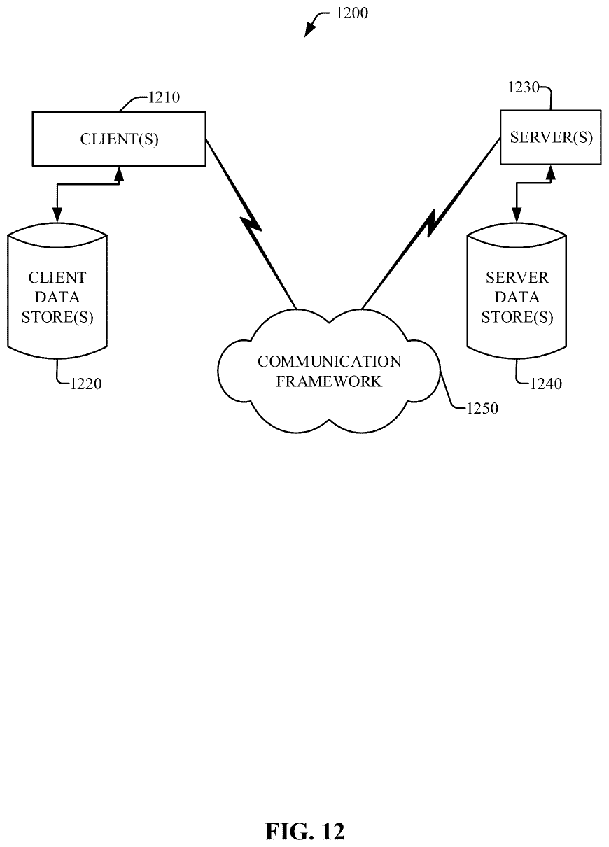

In order to provide a context for the various aspects of the disclosed subject matter, FIGS. 11 and 12 as well as the following discussion are intended to provide a brief, general description of a suitable environment in which the various aspects of the disclosed subject matter may be implemented.

With reference to FIG. 11, a suitable environment 1100 for implementing various aspects of this disclosure includes a computer 1112. The computer 1112 includes a processing unit 1114, a system memory 1116, and a system bus 1118. The system bus 1118 couples system components including, but not limited to, the system memory 1116 to the processing unit 1114. The processing unit 1114 can be any of various available processors. Dual microprocessors and other multiprocessor architectures also can be employed as the processing unit 1114.

The system bus 1118 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1116 includes volatile memory 1120 and nonvolatile memory 1122. The basic input/output system (BIOS), containing the basic routines to transfer information between elements within the computer 1112, such as during start-up, is stored in nonvolatile memory 1122. By way of illustration, and not limitation, nonvolatile memory 1122 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 1120 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 1112 also includes removable/non-removable, volatile/non-volatile computer storage media. FIG. 11 illustrates, for example, a disk storage 1124. Disk storage 1124 includes, but is not limited to, devices like a magnetic disk drive, floppy disk drive, tape drive, Jaz drive, Zip drive, LS-100 drive, flash memory card, or memory stick. The disk storage 1124 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 1124 to the system bus 1118, a removable or non-removable interface is typically used, such as interface 1126.

FIG. 11 also depicts software that acts as an intermediary between users and the basic computer resources described in the suitable operating environment 1100. Such software includes, for example, an operating system 1128. Operating system 1128, which can be stored on disk storage 1124, acts to control and allocate resources of the computer system 1112. System applications 1130 take advantage of the management of resources by operating system 1128 through program modules 1132 and program data 1134, e.g., stored either in system memory 1116 or on disk storage 1124. It is to be appreciated that this disclosure can be implemented with various operating systems or combinations of operating systems.

A user enters commands or information into the computer 1112 through input device(s) 1136. Input devices 1136 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1114 through the system bus 1118 via interface port(s) 1138. Interface port(s) 1138 include, for example, a serial port, a parallel port, a game port, and a universal serial bus (USB). Output device(s) 1140 use some of the same type of ports as input device(s) 1136. Thus, for example, a USB port may be used to provide input to computer 1112, and to output information from computer 1112 to an output device 1140. Output adapter 1142 is provided to illustrate that there are some output devices 1140 like monitors, speakers, and printers, among other output devices 1140, which require special adapters. The output adapters 1142 include, by way of illustration and not limitation, video and sound cards that provide a means of connection between the output device 1140 and the system bus 1118. It should be noted that other devices and/or systems of devices provide both input and output capabilities such as remote computer(s) 1144.

Computer 1112 can operate in a networked environment using logical connections to one or more remote computers, such as remote computer(s) 1144. The remote computer(s) 1144 can be a personal computer, a server, a router, a network PC, a workstation, a microprocessor based appliance, a peer device or other common network node and the like, and typically includes many or all of the elements described relative to computer 1112. For purposes of brevity, only a memory storage device 1146 is illustrated with remote computer(s) 1144. Remote computer(s) 1144 is logically connected to computer 1112 through a network interface 1148 and then physically connected via communication connection 1150. Network interface 1148 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1150 refers to the hardware/software employed to connect the network interface 1148 to the bus 1118. While communication connection 1150 is shown for illustrative clarity inside computer 1112, it can also be external to computer 1112. The hardware/software necessary for connection to the network interface 1148 includes, for exemplary purposes only, internal and external technologies such as, modems including regular telephone grade modems, cable modems and DSL modems, ISDN adapters, and Ethernet cards.

FIG. 12 is a schematic block diagram of a sample-computing environment 1200 with which the subject matter of this disclosure can interact. The system 1200 includes one or more client(s) 1210. The client(s) 1210 can be hardware and/or software (e.g., threads, processes, computing devices). The system 1200 also includes one or more server(s) 1230. Thus, system 1200 can correspond to a two-tier client server model or a multi-tier model (e.g., client, middle tier server, data server), amongst other models. The server(s) 1230 can also be hardware and/or software (e.g., threads, processes, computing devices). The servers 1230 can house threads to perform transformations by employing this disclosure, for example. One possible communication between a client 1210 and a server 1230 may be in the form of a data packet transmitted between two or more computer processes.

The system 1200 includes a communication framework 1250 that can be employed to facilitate communications between the client(s) 1210 and the server(s) 1230. The client(s) 1210 are operatively connected to one or more client data store(s) 1220 that can be employed to store information local to the client(s) 1210. Similarly, the server(s) 1230 are operatively connected to one or more server data store(s) 1240 that can be employed to store information local to the servers 1230.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2 (3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

### EXAMPLE ASPECTS

It is appreciated that aspects detailed herein can generally be combined together in any suitable combination. In some cases, if relevant, any aspect noted below can be combined with any other aspect.

Aspect 1. A system or device, comprising: a memory that stores computer executable components; and a processor that executes computer executable components stored in the memory, wherein the computer executable components comprise: a protocoling component that receives a medical examination order request in a standardized input format and, based on a machine learning technique, outputs a recommended protocol according to a standardized output format; and a generalizable component that performs a mapping procedure that maps site-specific data to the standardized input format and the standardized output format, wherein the site-specific data comprises information that is specific to an entity that provides the medical examination order request.

Aspect 2. The system or device in accordance with aspect 1, wherein the recommended protocol is selected in response to the recommended protocol having a highest confidence score.

Aspect 3. The system or device in accordance with aspect 1, wherein the recommended protocol comprises a group of recommended protocols having respective confidence scores that are determined to be above a defined threshold.

Aspect 4. The system or device in accordance with aspect 1, further comprising a decision support component that, in response to examining medical data, determines a decision support indicator that indicates whether a protocoler is notified to review recommended protocol.

Aspect 5. The system or device in accordance with aspect 1 or 4, wherein, in response to the recommended protocol comprising a group of recommended protocols, the decision support component determines the decision support indicator is to indicate the protocoler is to be notified to review the recommended protocol.

Aspect 6. The system or device in accordance with aspect 1 or 4, wherein the medical data comprises an electronic health record associated with a patient identified by the medical examination order request and the decision support component determines the decision support indicator is to indicate the protocoler is to be notified to review the recommended protocol in response to rules-based examination of the electronic health record.

Aspect 7. The system or device in accordance with aspect 1 or 4, wherein the decision support component determines the decision support indicator is to indicate the protocoler is to be notified to review the recommended protocol in response to the recommended protocol having a confidence score below a defined threshold.

Aspect 8. The system or device in accordance with aspect 1 or 4, wherein the decision support component determines the decision support indicator is to indicate the protocoler is to be notified to review the recommended protocol in response to the generalizable component failing to map the recommended protocol to a site-specific protocol.

Aspect 9. The system or device in accordance with aspect 1, wherein the mapping procedure maps the site-specific data to the standardized input format and the standardized output format according to a rules-based procedure that employs defined rules to associate site-specific protocols included in the site-specific data to standardized protocols included in the standardized input formats and the standardized output formats.

Aspect 10. The system or device in accordance with aspect 1, further comprising an update component that periodically requests updates to the site-specific data.

Aspect 11. The system or device in accordance with aspect 1, wherein the mapping procedure maps the site-specific data to the standardized input format and the standardized output format according to a machine learning classifier that is trained, via a learning procedure, to classify site-specific protocols to standardized protocols.

Aspect 12. The system or device in accordance with aspect 1, wherein the learning procedure trains the machine learning classifier based on input that identifies a protocol to satisfy the medical examination order request.

Aspect 13. The system or device in accordance with aspect 1, further comprising a machine-level protocol component that, based on the recommended protocol, recommends a machine procedure specific to a device used to satisfy the medical examination order request.

Aspect 14. A method, comprising: receiving, by a system operatively coupled to a processor, a medical order examination request; determining, by the system, a recommended protocol to employ to satisfy the medical examination order request based on a machine learning technique; and mapping, by the system, the recommended protocol from a standardized format suitable for the machine learning technique to a site-specific format associated with an entity that provides the medical order examination request.

Aspect 15. The method in accordance with aspect 14, further comprising employing, by the system, a rules-based engine to map the recommended protocol from the standardized format to the site-specific format based on site-specific data.

Aspect 16. The method in accordance with aspect 14 or 15, further comprising requesting, by the system, an update to the site-specific data based on a defined schedule.

Aspect 17. The method in accordance with aspect 14, further comprising employing, by the system, a machine learning classifier to map the recommended protocol from the standardized format to the site-specific format, wherein the machine learning classifier is trained according to a learning procedure.

Aspect 18. A non-transitory machine-readable storage medium, comprising executable instructions that, when executed by a processor, facilitate performance of operations, comprising: receiving a medical order examination request; determining a recommended protocol to employ to satisfy a medical examination order request based on a machine learning technique; and mapping the recommended protocol from a standardized format suitable for the machine learning technique to a site-specific format associated with an entity that provides the medical order examination request.

Aspect 19. The non-transitory machine-readable storage medium in accordance with aspect 18, wherein the operations further comprise, employing a rules-based engine to map the recommended protocol from the standardized format to the site-specific format based on site-specific data.

Aspect 20. The non-transitory machine-readable storage medium in accordance with aspect 18, wherein the operations further comprise, employing a machine learning classifier to map the recommended protocol from the standardized format to the site-specific format, wherein the machine learning classifier is trained according to a learning procedure.

1. A system, comprising:
a memory that stores computer executable components; and
a processor that executes computer executable components stored in the memory, wherein the computer executable components comprise:
a protocoling component that receives a medical examination order request in a standardized input format and, based on a machine learning technique, outputs a recommended protocol according to a standardized output format; and
a generalizable component that performs a mapping procedure that maps site-specific data to the standardized input format and the standardized output format, wherein the site-specific data comprises information that is specific to an entity that provides the medical examination order request.

2. The system of claim 1, wherein the recommended protocol is selected in response to the recommended protocol having a highest confidence score.

3. The system of claim 1, wherein the recommended protocol comprises a group of recommended protocols having respective confidence scores that are determined to be above a defined threshold.

4. The system of claim 1, further comprising a decision support component that, in response to examining medical data, determines a decision support indicator that indicates whether a protocoler is notified to review recommended protocol.

5. The system of claim 4, wherein, in response to the recommended protocol comprising a group of recommended protocols, the decision support component determines the decision support indicator is to indicate the protocoler is to be notified to review the recommended protocol.

6. The system of claim 4, wherein the medical data comprises an electronic health record associated with a patient identified by the medical examination order request and the decision support component determines the decision support indicator is to indicate the protocoler is to be notified to review the recommended protocol in response to rules-based examination of the electronic health record.

7. The system of claim 4, wherein the decision support component determines the decision support indicator is to indicate the protocoler is to be notified to review the recommended protocol in response to the recommended protocol having a confidence score below a defined threshold.

8. The system of claim 4, wherein the decision support component determines the decision support indicator is to indicate the protocoler is to be notified to review the recommended protocol in response to the generalizable component failing to map the recommended protocol to a site-specific protocol.

9. The system of claim 1, wherein the mapping procedure maps the site-specific data to the standardized input format and the standardized output format according to a rules-based procedure that employs defined rules to associate site-specific protocols included in the site-specific data to standardized protocols included in the standardized input formats and the standardized output formats.

10. The system of claim 1, further comprising an update component that periodically requests updates to the site-specific data.

11. The system of claim 1, wherein the mapping procedure maps the site-specific data to the standardized input format and the standardized output format according to a machine learning classifier that is trained, via a learning procedure, to classify site-specific protocols to standardized protocols.

12. The system of claim 1, wherein the learning procedure trains the machine learning classifier based on input that identifies a protocol to satisfy the medical examination order request.

13. The system of claim 1, further comprising a machine-level protocol component that, based on the recommended protocol, recommends a machine procedure specific to a device used to satisfy the medical examination order request.

14. A method, comprising:
receiving, by a system operatively coupled to a processor, a medical order examination request;
determining, by the system, a recommended protocol to employ to satisfy the medical examination order request based on a machine learning technique; and
mapping, by the system, the recommended protocol from a standardized format suitable for the machine learning technique to a site-specific format associated with an entity that provides the medical order examination request.

15. The method of claim 14, further comprising employing, by the system, a rules-based engine to map the recommended protocol from the standardized format to the site-specific format based on site-specific data.

16. The method of claim 15, further comprising requesting, by the system, an update to the site-specific data based on a defined schedule.

17. The method of claim 14, further comprising employing, by the system, a machine learning classifier to map the recommended protocol from the standardized format to the site-specific format, wherein the machine learning classifier is trained according to a learning procedure.

18. A non-transitory machine-readable storage medium, comprising executable instructions that, when executed by a processor, facilitate performance of operations, comprising:
receiving a medical order examination request;
determining a recommended protocol to employ to satisfy a medical examination order request based on a machine learning technique; and
mapping the recommended protocol from a standardized format suitable for the machine learning technique to a site-specific format associated with an entity that provides the medical order examination request.

19. The non-transitory machine-readable storage medium of claim 18, wherein the operations further comprise, employing a rules-based engine to map the recommended protocol from the standardized format to the site-specific format based on site-specific data.

20. The non-transitory machine-readable storage medium of claim 18, wherein the operations further comprise, employing a machine learning classifier to map the recommended protocol from the standardized format to the site-specific format, wherein the machine learning classifier is trained according to a learning procedure.
